(19) United States
(12) Patent Application Publication
(10) Pub. No.: US 2026/0240436 A1
(43) Pub. Date: Aug. 20, 2026

(54) INTRAVASCULAR MECHANICAL SHAPE AND STIFFNESS SENSOR

(71) Applicant: KONINKLIJKE PHILIPS N.V., EINDHOVEN (NL)

(72) Inventors: Rene Leonardus Jacobus Marie UBACHS, EINDHOVEN (NL); Leili SALEHI, WALTHAM, MA (US); Vipul Shrihari PAI RAIKAR, SOMERVILLE, MA (US); Alyssa TORJESEN, EXETER, MA (US); Bernardus Hendrikus Wilhelmus HENDRIKS, EINDHOVEN (NL)

(21) Appl. No.: 19/164,628

(22) PCT Filed: Mar. 4, 2024
(86) PCT No.: PCT/EP2024/055526

§ 371 Date: Sep. 12, 2025

Related U.S. Application Data
(60) Provisional application No. 63/451,963, filed on Mar. 14, 2023.

(51) Int. Cl. A61B 5/02; A61B 1/05; A61B 5/0538

(52) U.S. Cl. CPC A61B 5/02007; A61B 1/05; A61B 5/0538; A61B 2562/0261

(57) ABSTRACT

A device for intravascular assessment includes a catheter (12) configured for insertion into a blood vessel (V); a plurality of feelers (14) extending circumferentially outward around a distal end of the catheter and configured to press against a wall of the blood vessel in which the catheter is inserted; and a sensor (16) configured to acquire data on at least one of: a shape formed by the feelers pressing against the wall of the blood vessel and a force applied by the feelers pressing against the wall of the blood vessel.

### FIELD

The following relates generally to the intravascular therapy instrument arts, blood vessel property measurement arts, intravascular therapy sensor arts, and related arts.

### BACKGROUND

Treatment of obstructed arteries or veins may involve angioplasty, stenting, and/or other intravascular therapies in which a catheter is used to deliver a therapy instrument to the site of the obstruction. For optimal therapy planning, the geometry of the stenosed vessel is valuable information. It is used to decide on the size of angioplasty balloon, the ballooning pressure, and the duration of applying pressure, or similarly to decide the size and other properties of a stent. This information can be obtained through an imaging modality such as two-dimensional (2D) fluoroscopy, computed tomography (CT), or intravascular ultrasound (IVUS). However, CT is expensive and may be of insufficient resolution to capture the geometry of the stenosed vessel in sufficient detail. 2D fluoroscopy provides 2D images which may be insufficient to determine the three-dimensional (3D) geometry of the stenosed vessel. IVUS is invasive and also may fail to provide sufficient information to satisfactorily determine the 3D geometry of the stenosed blood vessel.

Additional information on the tissue properties of the blood vessel is very welcomed for therapy planning; for example, the amount and location of calcifications and plaque. This information can also be obtained from imaging modalities. However, mechanical properties cannot be accurately obtained by imaging techniques. These properties (e.g., elasticity of the vessel wall, hardness/stiffness of the calcifications, etc.) are critical when estimating the effect that a certain treatment will have on the final state of the (un)stented vessel.

The following discloses certain improvements to overcome these problems and others.

### SUMMARY

In some embodiments disclosed herein, a device for intravascular assessment includes a catheter configured for insertion into a blood vessel; a plurality of feelers extending circumferentially outward around a distal end of the catheter and configured to press against a wall of the blood vessel in which the catheter is inserted; and a sensor configured to acquire data on at least one of: (i) a shape formed by the feelers pressing against the wall of the blood vessel and (ii) a force applied by the feelers pressing against the wall of the blood vessel.

In some embodiments disclosed herein, a method of characterizing a wall of a blood vessel includes at least partially withdrawing a catheter inserted into a blood vessel, the catheter having a plurality of feelers extending circumferentially outward around a distal end of the catheter and pressing against the wall of the blood vessel during the withdrawing; during the withdrawing, acquiring at least one of data on a shape of the feelers and data on a force applied by the feelers pressing against the wall of the blood vessel; and determining at least one of a shape of the wall of the blood vessel and a stiffness of the wall of the blood vessel based on the acquired data.

One advantage resides in providing an intravascular device configured for direct determination of the shape and mechanical properties of the vessel though direct or indirect measurement.

Another advantage resides in providing an intravascular device configured to assess severity of stenosis and/or determine severity of arteriosclerosis.

Another advantage resides in providing an intravascular device configured for determination of a stiffness of the vessel.

Another advantage resides in providing data related to a size and mechanical properties of the vessel to determine an intravascular therapy device for placement therein.

Another advantage resides in reducing an amount of time a patient needs to be imaged.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a system with an intravascular therapy or atherectomy device in accordance with the present disclosure.

FIGS. 2A and 2B diagrammatically illustrates another embodiment of the device of FIG. 1.

FIGS. 3A and 3B diagrammatically illustrates another embodiment of the device of FIG. 1.

FIGS. 4A and 4B diagrammatically illustrates another embodiment of the device of FIG. 1.

FIG. 5 diagrammatically illustrates a method of characterizing a vessel wall using one of the devices of FIGS. 1-4B.

FIGS. 6A, 6B, and 6C diagrammatically illustrate the device of FIG. 1 in use.

### DETAILED DESCRIPTION

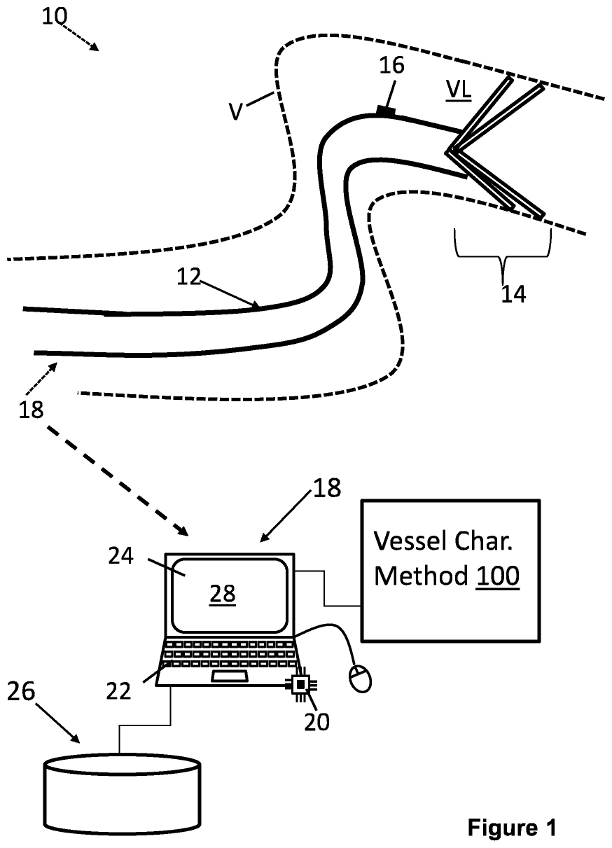

FIG. 1 illustrates a system 50 in an embodiment of the invention. With reference to FIG. 1, a distal end of an illustrative intravascular device 10 of the system 50 is shown inserted into a blood vessel V (drawn using dashed lines) having a vessel lumen VL. As shown in FIG. 1, the device 10 includes a catheter 12. A plurality of feelers 14 (diagrammatically shown in FIG. 1) extend circumferentially outward around a distal end of the catheter 12. To initiate intravascular assessment of a blood vessel wall and clot, occlusion, or other build-up disposed thereon using the intravascular device 10, the distal end of the catheter 12 is inserted into a blood vessel (e.g., a vein in preparation for a thrombectomy procedure, or an artery in preparation for an atherectomy procedure) through an incision accessing a blood vessel made by the surgeon. A length of the catheter 12 is then pushed through the blood vessel until the tip of the catheter 12 is positioned past the treatment site. This may optionally be done with the aid of a medical imaging modality, such as ultrasound imaging or computed tomography (CT) imaging to guide the surgeon in placement of the catheter tip. Thereafter, the feelers 14 are deployed by being extended out of a recess or lumen at the distal end of the catheter 12, and then the catheter 12 is withdrawn so that the circumferentially outwardly extended feelers 14 are drawn across and circumferentially contact the treatment site as the tip of the catheter 12 is drawn back through the treatment site.

The feelers 14 can comprise a deformable mechanical (near) circular structure with known mechanical behaviors and a diameter (when fully outwardly expanded) that is larger than the vessel V and configured to be moved (e.g., by pulling) through the vessel V. The feelers 14 thus are arranged to press against a wall of a blood vessel V in which the catheter 12 is disposed, and the tips of the feelers 14 drag against the wall of the blood vessel V as the catheter is withdrawn. In some embodiments, the feelers 14 comprise wires, while in other embodiments, the feelers 14 comprise leaf springs, as two nonlimiting illustrative examples. The leaf springs can effectively reduce the freedom of movement of the feelers 14, leading to more control and facilitating a lumen shape construction algorithm. The feelers 14 are configured to be expanded from, and retracted into, and stowed in the distal end of the catheter 12.

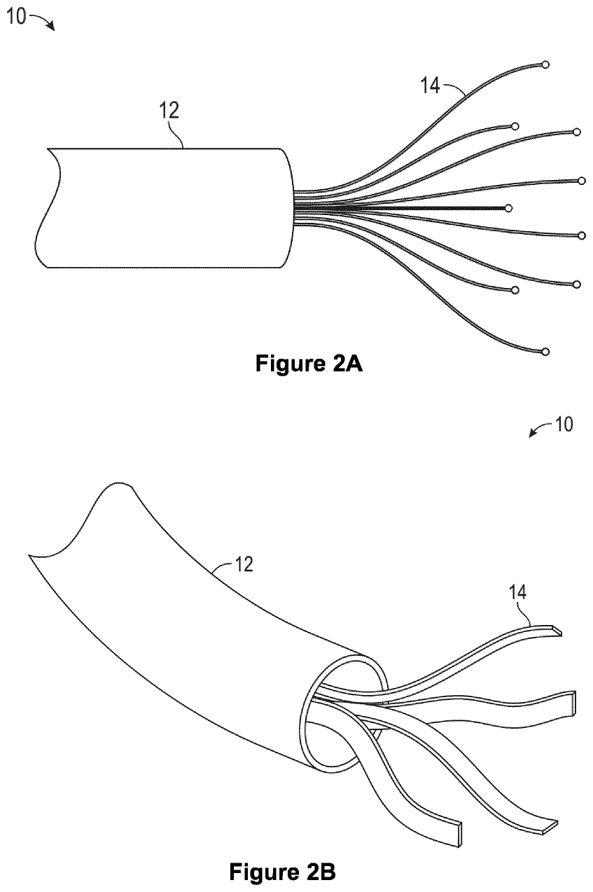

With continuing reference to FIG. 1, and now referring to FIGS. 2A and 2B, the shape of the structure of the feelers 14 is changed due to its interaction with the vessel V. FIG. 2A shows an example of the feelers 14 as wires, while FIG. 2B shows an example of the feelers 14 as leaf springs. By monitoring the shape and position of the feelers 14, as they drag across the treatment site as the catheter 12 is withdrawn, a three-dimensional (3D) reconstruction of an interior of the vessel V can be made. Based on knowledge of the mechanical behavior and geometry of the structure of the feelers 14 at various locations in the vessel V, corresponding forces exerted by the vessel V on the feelers 14 can be calculated. The procedure is repeated with the same structure with an adapted stiffness (for example, by changing the effective length of the feelers 14 by changing the length of their extension out of the distal end of the catheter 12) leading to a second reconstructed shape. The procedure may also be repeated with a second structure with a different, but known, stiffness and/or geometry. The difference between the first and second reconstruction, in combination with knowledge on the forces applied in the two cases, allows for the creation of a vessel stiffness map. Improved stiffness mapping accuracy may, optionally, be obtained by further repeating for additional feeler stiffness values (e.g., obtained by different extended feeler lengths).

The feelers 14 may be made of a biocompatible material with sufficient pliability to deform and accurately track the shape of the treatment site without damaging the tissue of the blood vessel wall. Some suitable materials include stainless steel wires or leaf springs, plastic feelers, or so forth. In some embodiments, the feelers 14 are made of a biocompatible pseudoelastic shape memory nitinol (an alloy of nickel and titanium) or another shape memory material exhibiting pseudoelasticity that is set in the fully outwardly expanded configuration and, thus, can be compacted into a recess at the distal end of the catheter 12 for delivery and upon deployment by extension out of the recess expand to their fully outwardly expanded configuration (constrained however by the space provided by the lumen VL of the blood vessel V).

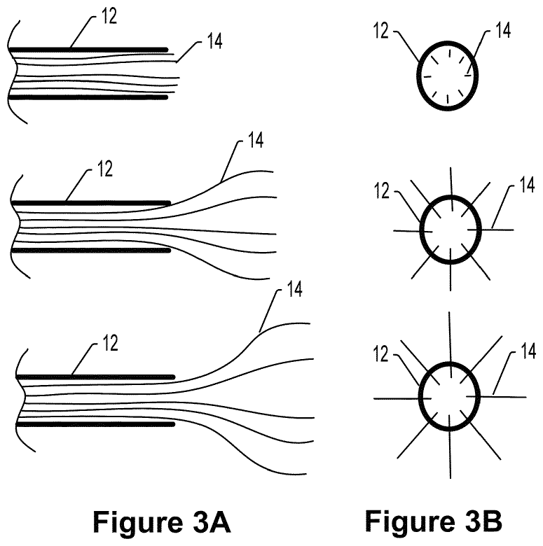

FIG. 3A shows a progression of side views of deployment of the feelers 14 from the catheter 12, and FIG. 3B shows the progression of corresponding front views of the deployment of the feelers 14 from the catheter 12. The feelers 14 are shown as a circular array of pre-bent wires which can be pulled into the catheter 12. This action will straighten the wires 14 temporarily for the duration of its time in the catheter 12. This is the deployment configuration, in which the feelers 14 are retracted into the distal end of the catheter 12, so that they do not interfere with movement of the catheter 12 through the blood vessel as it is pushed toward and past the treatment site. When the distal end of the catheter 12 is past the treatment site, the feelers 14 are deployed by being pushed out. When pushed out, the radius of the array of the feelers 14 (measured at the ends/tips) will increase. When pushed out further, the diameter of the structure of the feelers 14 may increase or may be kept constant, depending on the pre-shaping of the feelers 14. Either way, the stiffness of the structure of the feelers 14 will change. Typically, the feelers 14 will become less stiff the further out they are pushed. For example, the feelers 14 in the more extended position of the lower drawing of FIG. 3A are less stiff than the feelers 14 in the less extended position of the middle drawing of FIG. 3A). The position of the tips of the feelers 14 is continuously monitored, e.g., using an intravascular sensor and/or by imaging. In this way, the shape of the lumen VL of the blood vessel V at the position of the feelers 14 can be constructed.

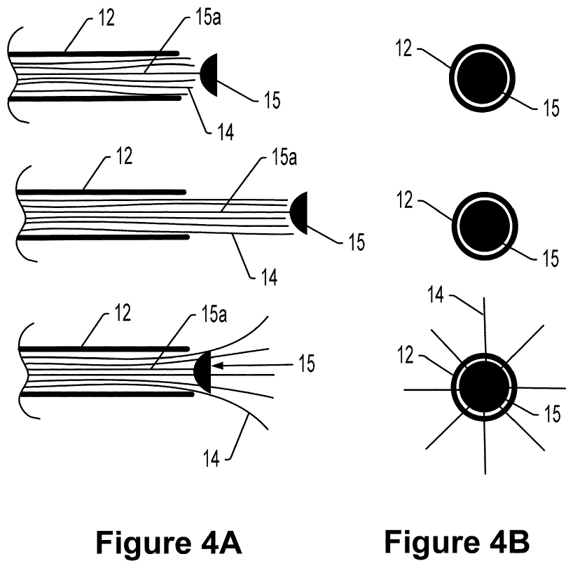

FIG. 4A shows a progression of side views of deployment of the feelers 14 according to another embodiment from the catheter 12. This embodiment further includes a mechanism 15 (shown as a spherical or wedge-shaped mechanism), and FIG. 4B shows the progression of corresponding front views of the deployment of the feelers 14 from the catheter 12 with the mechanism 15. As shown in FIGS. 4A and 4B, the feelers 14 are initially straight, and a wire or other tether 15a in the center is connected to the spherical or wedge-shaped mechanism 15. The mechanism 15 is initially placed distal to the tips of the feelers 14 and can be pulled proximally, thus, forcing the feelers 14 to bend outward. The mechanism 15 between the feelers 14 can control a maximum diameter of a structure of the feelers 14. This may be beneficial to pass bifurcations and side branches more easily and safely in the vessel V. The mechanism 15 can also be positioned proximal and/or distal to the feelers 14 to open the lumen of the blood vessel V with a specific diameter and/or force, allowing a user to better evaluate a state of lumen after intravascular therapy device placement. This embodiment may also be used to locally determine the stiffness of the vessel V. By keeping the catheter in place and changing the stiffness of the feelers through repositioning of the mechanism 15, the corresponding geometry of the feelers 14 then allows for determination of local vessel stiffness. In another embodiment, two or more sets of feelers 14 with different stiffnesses are placed on a single catheter 12 with a suitable distance in between. In this way, all necessary information for determination of vessel shape and stiffness may be obtained with a single pass of the device 10.

Referring back to FIG. 1, the device 10 also includes a sensor 16 disposed at the distal end of the catheter 12 and configured to acquire data on a shape formed by the feelers pressing against the wall of the blood vessel V and/or data on force applied by the feelers 14 pressing against the wall of the blood vessel V. Although the sensor 16 is shown at the distal end of the catheter 12, the sensor 16 can be positioned on any portion of the device 10. The sensor 16 can be implemented in a variety of suitable ways.

In some embodiments, the sensor 16 comprises an impedance sensor comprising electrodes disposed on the feelers 16. In one example, the impedance sensor 16 is configured to determine a distance of each feeler 14 relative to the catheter 12. In another example, the impedance sensor 16 is configured to determine an electrical field between multiple feelers 14. In another example, the electrodes of the impedance sensor 16 are positioned toward tips of the feelers 14 through which the feeler tip position can be tracked using electrical impedance. In another example, the electrodes of the impedance sensor 16 are positioned on the vessel V (possibly in contact with the tissue) and are used to measure the impedance of the tissue. Based on measurements of the impedance using a frequency sweep, tissue properties of the vessel V can be derived. In another example, an electrode of the impedance sensor 16 is positioned in a center of the feelers 14 to measure a distance from a center of the feelers 14 to the individual feelers 14.

In other embodiments, the sensor 16 comprises a strain gauge operatively connected to the feelers 14 and configured to measure a strain of the feelers 14. From the measured strains, the geometry of the deformed feelers 14 may be calculated.

In some embodiments, the sensor 16 comprises one or more fiber optic real-shape (FORS) wires integrated with the feelers 14 and configured to determine the shape and/or position of the feelers 14. For example, each feeler 14 can be a FORS wire, or can have a FORS wire integrated therewith, to monitor the shape of the feeler.

In other embodiments, the sensor 16 comprises an intravascular ultrasound (IVUS) sensor that includes an imaging device configured to acquire an image of the feelers 14, and an image processor (i.e., a hardware or computer processor) of the system 50 is programmed to determine the shape of the feelers 14 from the image.

These are merely examples and should not be construed as limiting.

FIG. 1 also shows an electronic processor 18 of the system 50 programmed (configured) to determine a shape and/or stiffness of the wall of the blood vessel V based on the data acquired by the sensor 16. In FIG. 1, the electronic processor 18 is separate from the catheter 12 and device 10. To provide operative connection to receive the acquired data from the sensor 16, the sensor 16 is operatively connected with the electronic processor 18 wirelessly or by wires passing along the catheter 12 from its proximal end outside of the patient to its distal end located inside the patient at about the treatment site. In other embodiments, the electronic processor 18 can be integrated with the catheter 12, e.g., positioned on a portion of the device 10. In some embodiments, the sensor 16 is configured to acquire data on the force applied by the feelers 14 pressing against the wall of the blood vessel V, and the electronic processor 18 is programmed to determine the stiffness of the wall of the blood vessel V based on the acquired data on the force applied by the feelers 14. To do so, in some embodiments, the feelers 14 are configured to have adjustable stiffness, and the electronic processor 18 is programmed to determine the stiffness of the wall of the blood vessel V based on the data on the force applied by the feelers 14 acquired for at least two different values of the adjustable stiffness of the feelers 14. In some nonlimiting illustrative examples, the stiffness may be adjusted by adjusting the extended length of the feelers 14 out of the distal end of the catheter 12, as in the embodiment of FIGS. 3A and 3B, or the stiffness may be adjusted by moving the wedge-shaped mechanism 15 in the embodiment of FIGS. 4A and 4B.

In some embodiments, the electronic processor 18 are part of an electronic processing device 18 operatively connected with the sensor 16. The electronic processing device 18 can comprise any processing device, such as a workstation computer, or more generally a computer. The electronic processing device 18 may also include a server computer or a plurality of server computers, e.g., interconnected to form a server cluster, cloud computing resource, or so forth, to perform more complex computational tasks. The electronic processing device 18 includes typical components, such as an electronic processor 20 (e.g., a microprocessor), at least one user input device (e.g., a mouse, a keyboard, a trackball, and/or the like) 22, and a display device 24 (e.g., an LCD display, plasma display, cathode ray tube display, and/or so forth). In some embodiments, the display device 24 can be a separate component from the electronic processing device 18 or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the electronic processing device 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions may include instructions to generate a visualization of a graphical user interface (GUI) 28 for display on the display device 24.

The processor 18 is configured as described above to perform one or more operations of a method or process 100 for characterizing the wall of a blood vessel. The non-transitory storage medium 26 stores instructions which are readable and executable by the at least one electronic processor 20 to perform disclosed operations including performing the vascular therapy method or process 100. In some examples, the method 100 may be performed at least in part by cloud processing.

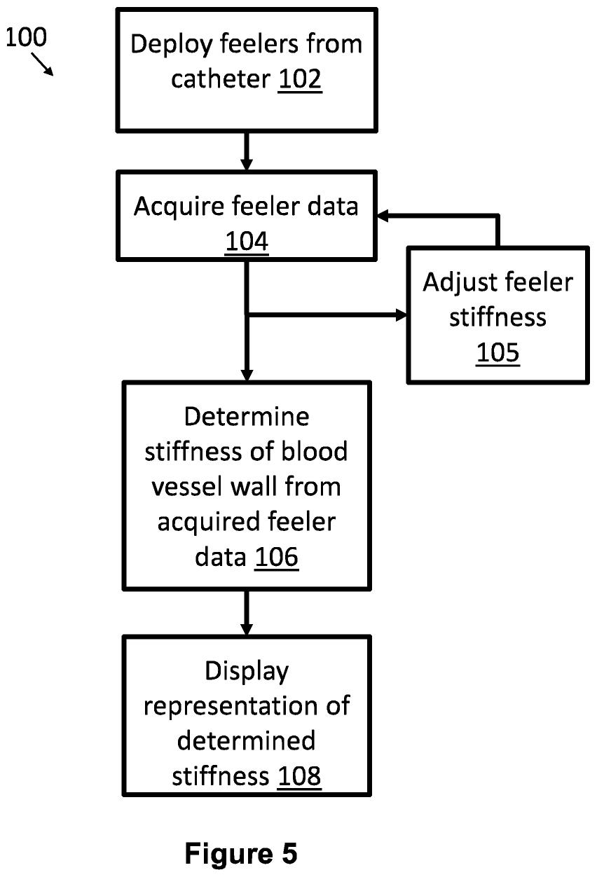

FIG. 5 illustrates a flowchart showing an example method 100 of characterizing the wall of a blood vessel V using the device 10 of FIG. 1, in an embodiment of the present invention. To begin the method 100, the catheter 12 is inserted into the blood vessel V. At an operation 102, the catheter 12 is at least partially withdrawn from the blood vessel V while the catheter 12 presses against a wall of the blood vessel V during the withdrawing. By withdrawing the catheter 12, the feelers 14 are deployed from the catheter 12 to contact and press against the wall of the blood vessel V. During the withdrawing, the feelers 14 trail the distal end of the catheter 12.

At an operation 104, during the withdrawing of the catheter 12, the sensor 16 is configured to acquire data on a shape of the feelers 14 and/or data on force applied by the feelers 14 pressing against the wall of the blood vessel V.

A single execution of the operation 104 with a single withdrawal pass of the catheter may acquire sufficient data from the sensor 16 to determine the shape of the treatment site three-dimensionally. However, for measuring tissue stiffness, two or more passes of the operation 104 with different feeler stiffness values may be used. To this end, FIG. 5 includes a loopback operation 105 in which the stiffness of the feelers 14 is adjusted. The loop 104, 105 may be iterated once to obtain sensor data for two different stiffnesses or may be further iterated to obtain sensor data for three (or more) different stiffnesses.

At an operation 106, the electronic processor 18 is configured to determine a shape and/or stiffness of the wall of the blood vessel V based on the acquired data. For example, an extension of the feelers 14 from the distal end of the catheter 12 is adjustable, and a stiffness of the feelers 14 depends on how far the feelers are extended out of the distal end of the catheter 12 during the withdrawing operation 102. During the acquiring operation 104, the acquiring of the data on the shape of the feelers 14 and/or the data on the force applied by the feelers 14 pressing against the wall of the blood vessel V is performed for at least two different stiffnesses of the feelers 14 via the loopback feeler stiffness adjustment 105. The determining operation 106 then includes determining the stiffness of the wall of the blood vessel V based on the acquired data at the at least two different stiffnesses of the feelers 14.

At an optional operation 108, the electronic processing device 18 is configured to generate a representation of the stiffness V of the wall of the blood vessel V. Such a representation can be displayed on the display device 24. In some embodiments, the operation 108 may display a 3D rendering of the geometry of the treatment site. In some embodiments, the operation 108 may display tissue of the treatment site with the measured tissue stiffness indicated by color coding or the like. These are merely nonlimiting illustrative examples.

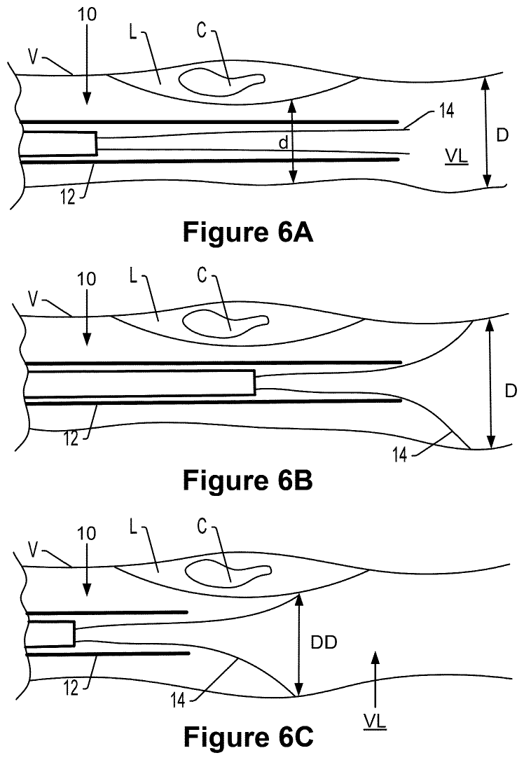

FIGS. 6A-6C show an example method 100 using the device 10 to perform the data acquisition operation 104 of FIG. 5. As shown in FIG. 6A, the device 10 is introduced into a vessel lumen VL and navigated to a lesion site L. As shown in FIG. 6B, the feelers 14 are deployed from the distal end of the catheter 12, such that they make contact with the wall of the blood vessel V. Upon the feelers 14 making contact with the vessel wall, the shape of the vessel V changes (characterized by the change of a first diameter d to a second larger dimension D). The magnitude of this change is determined by the vessel properties and the stiffness of the feelers 14. Upon proximal movement of the device 10 through the vessel V, a new position of the device-vessel contact is established. Here, a new force equilibrium between the vessel V and the device 10 is established, leading to a changed vessel and device size.

In case the vessel V has a higher stiffness (i.e., is less compliant), the device 10 is forced to deform more (and vice versa). For example, in the region with stenosis due to a plaque deposition in the lesion L, calcification C is present, resulting in a stiffening of the vessel wall. Placing the device 10 in at this position results in the lumen size to increase slightly from the first dimension d to a third dimension DD, but in a larger dimension decrease of the device 10 from the first dimension d to the third dimension DD.

Performing the above procedure one time results in the determination of a point in a force versus lumen size curve, which can be displayed on the display device 14. Repeating the procedure with a changed stiffness of the device results in a second point allowing the determination of the vessel stiffness at these load levels.

As biological material properties are generally non-linear, the procedure may be repeated with different force levels and/or after angioplasty has been performed. The latter is not only due to non-linearity, but also because angioplasty will introduce damage in the tissues, altering the mechanical properties of the vessel V.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A device for intravascular assessment, the device comprising:
a catheter configured for insertion into a blood vessel
a plurality of feelers extending circumferentially outward around a distal end of the catheter and configured to press against a wall of the blood vessel that the catheter is inserted; and
a sensor configured to acquire data on at least one of: (i) a shape formed by the feelers pressing against the wall of the blood vessel and (ii) a force applied by the feelers pressing against the wall of the blood vessel.

2. The device of claim 1, wherein the plurality of feelers comprises a plurality of wires.

3. The device of claim 1, wherein the plurality of feelers comprises a plurality of leaf springs.

4. The device of claim 1, wherein the plurality of feelers are configured to be retracted into and stowed in the distal end of the catheter.

5. The device of claim 1, wherein the plurality of feelers are configured to have adjustable stiffness.

6. The device of claim 1, wherein the sensor comprises:
an impedance sensor comprising electrodes disposed on the feelers.

7. The device of claim 6, wherein the impedance sensor is configured to at least one of:
determine a distance of each of the plurality of feelers relative to the catheter and
determine an electrical field between multiple feelers of the plurality of feelers.

8. The device of claim 1, wherein the sensor comprises:
a strain gauge operatively connected to the plurality of feelers and configured to measure a strain of the plurality of feelers.

9. The device of claim 1, wherein the sensor comprises:
fiber optic real-shape (FORS) wires integrated with the plurality of feelers and configured to determine the shape of the plurality of feelers.

10. The device of claim 1, wherein the sensor comprises:
an imaging sensor configured to acquire an image of the plurality of feelers;

11. A system for intravascular assessment, the system comprising;
the device of claim 1; and
an electronic processor operatively connected to the device.

12. The system of claim 11, wherein the electronic processor is integrated with the device.

13. The system of claim 11, wherein the electronic processor is remote from the device and operatively connected with the sensor to receive the acquired data.

14. The system of claim 11, wherein the electronic processor is configured to at least one of:
determine the shape form by the feelers pressing against the wall of the blood vessel based on the data acquired by the sensor, and
determine the stiffness of the wall of the blood vessel based on the data acquired by the sensor.

15. The system of claim 11, further comprising:
an image processor configured to determine the shape of the plurality feelers based on an image acquired by the sensor.

16. The system of claim 11, wherein the electronic processor is configured to determine at least one of a shape of the wall of the blood vessel and a stiffness of the wall of the blood vessel based on the acquired data.

17. A method of characterizing a wall of a blood vessel, the method comprising:
at least partially withdrawing a catheter inserted into a blood vessel, the catheter having a plurality of feelers extending circumferentially outward around a distal end of the catheter and pressing against the wall of the blood vessel during the withdrawing;
during the withdrawing, acquiring at least one of data on a shape of the feelers and data on a force applied by the feelers pressing against the wall of the blood vessel; and
determining at least one of: a shape of the wall of the blood vessel and a stiffness of the wall of the blood vessel based on the acquired data.

18. The method of claim 17, wherein the feelers trail the distal end of the catheter during the withdrawing.

19. The method of claim 18, wherein:
an extension of the feelers from the distal end of the catheter is adjustable and a stiffness of the feelers depends on how far the feelers are extended out of the distal end of the catheter;
the acquiring of the at least one of the data on the shape of the feelers and the data on the force applied by the feelers pressing against the wall of the blood vessel is performed for at least two different stiffnesses of the feelers; and
the determining includes determining the stiffness of the wall of the blood vessel based on the acquired data at the at least two different stiffnesses of the feelers.

20. The method of claim 17, further including:
determining, with an impedance sensor disposed on the feelers, a distance of each of the plurality of feelers relative to the catheter.
